(19) United States
(12) Patent Application Publication
(10) Pub. No.: US 2022/0176097 A1
(43) Pub. Date: Jun. 9, 2022

(54) ANTI-THROMBTIC MEDICAL INSTRUMENT AND CATHETER

(71) Applicant: Ahmed Abdelaal AHMED MAHMOUD METWALLY ALKHATIP, BIRMINGHAM (GB)

(72) Inventor: Ahmed Abdelaal AHMED MAHMOUD METWALLY ALKHATIP, BIRMINGHAM (GB)

(21) Appl. No.: 17/436,153

(22) PCT Filed: Mar. 2, 2020
(86) PCT No.: PCT/IB2020/051747

§ 371 Date: Sep. 3, 2021

Related U.S. Application Data
(63) Continuation-in-part of application No. 16/267,766, filed on Feb. 5, 2019, now abandoned.

(51) Int. Cl. A61M 39/02; A61M 25/00; A61M 25/06

(52) U.S. Cl. CPC A61M 39/0247; A61M 25/007; A61M 25/0097; A61M 25/0606; A61M 2205/0216; A61M 2039/0258; A61M 2039/0264; A61M 2039/0282; A61M 2039/0291; A61M 25/0074

(57) ABSTRACT

The present invention relates to a an anti-thrombotic medical instrument and catheter medical instrument comprising: base with a port for connection to an infusion set; a catheter extending from the base, and comprises a main body of tubular configuration. a plurality of lateral conduits distributed along the length of the catheter; an elastomeric cover over each lateral conduit, said elastomeric cover opens with a fluid flow inside the catheter and closes when the fluid flow stops. Said lateral conduits have an angle in the range of 15° to 60° to the longitudinal axis of the catheter.

### FIELD OF INVENTION

The present invention relates to a medical instrument, particularly to an anti-thrombotic vascular retrograde flow catheter.

### BACKGROUND OF THE INVENTION

Over 5 million central venous or vascular catheters are inserted every year in the United States, according to The New England Journal of Medicine®. Catheters are often used in medical facilities for many patients who may need blood transfusions, fluids or medications, have cancer, heart or kidney problems and other issues. Using vascular catheters have high risks of many associated complications that result in morbidity, mortality, and increased healthcare cost. Usage of vascular catheters has been problematic because of coagulation, patients contracting bacterial infections, phlebitis and other infections. Fibrin sheath can form within 1 week of catheter placement and can occlude the distal openings resulting in an inability to infuse fluid or withdraw blood. After one day of catheter insertion, the catheter and the vein wall contact point begins to form fibrin sheath and then extends along the wall that can cause the growth of bacteria that leads to thrombosis or thrombophlebitis. The fibrin sheath accumulation on its surface can come from several factors such as the design of the catheter, the material used, and the position of the catheter's tip, increase the risk of catheter related thrombosis (CRT). Prevention of CRT with systemic anticoagulant prophylaxis has largely been ineffective. Many patients that had their catheter removed as a result of thrombosis had a series of complications after removal. It has been well known in the field to administer liquid into a patient's bloodstream by use of a central or peripheral vascular catheter. A typical vascular catheter device comprises a base with an injection port for connection to an infusion set, a catheter, and a metal trocar within the catheter. Insertion of a catheter into a vein is commonly performed in an antegrade direction, i.e. with the direction of blood flow. After the catheter and the metal trocar are inside the vessel, if the catheter is of the type which has a trocar, the catheter is pushed to slide over the trocar to lie within the lumen of the vessel, the trocar is removed, and the infusion set is connected to the catheter. Common problems associated with vascular cannulation or catheterization are infection and thrombosis. These two problems lead to frequent routine or mandatory change of the catheter. Thrombosis includes superficial and deep venous thrombosis and possibly pulmonary embolism, or arterial thrombosis with possible subsequent ischemia. Ultrasound assessment of catheterized vessels often shows a high risk of catheter-related thrombosis even in the absence of clinical signs of thrombosis and with a functioning vascular catheter. Catheter-related thrombosis usually starts at the angle between the catheter and the vessel wall, being an area of relative blood stagnation and stasis leading to the development of a thrombus. Catheter-related thrombus can propagate distally to the tip of the catheter, causing catheter obstruction and failure. Or, it can initiate superficial or deep venous thrombosis with possible pulmonary embolism. A catheter-related thrombosis region can become infected, being an area of blood stasis, and hence can lead to thrombophlebitis, which carries the risk of both sepsis and thrombosis. Therefore, there is a need for an effective method of preventing fibrin sheath resulting in thrombosis, bacterial formation, infection etc. and reduce burden for health care workers and help establish a better recovery period for patients being treated for various illnesses.

WO2009132065 discloses a peripheral catheter having a catheter tip diffuser for reducing an exit velocity of an infusant within the catheter. Pluralities of diffusion side holes are provided on the tip portion of the catheter.

EP patent EP2168625 Describes a valved hemodialysis catheter, which comprises: an elongated catheter member defining a longitudinal axis and at least one lumen for passage of fluid, and having proximal and distal end regions; and a valve disposed adjacent the distal end region of the catheter member, the valve adapted to move from a closed position to an open position in response to a predetermined vacuum pressure level within the at least one lumen to permit flow of the fluid through the valve and into the at least one lumen.

However, the solutions mentioned above do not provide an effective solution to the blood stasis and thrombosis in the peri-catheter region.

It is therefore an object of the present invention, to provide an anti-thrombotic medical instrument and catheter.

### SUMMARY OF THE INVENTION

Various embodiments of medical instrument including a catheter are disclosed. Distinguishing features that may be included in this medical instrument are described below. It is intended that the medical instrument may include one or more of these features individually or in combination and it is not intended that the medical instrument be limited to the specific embodiments described herein.

Accordingly, the present invention relates to further improvements in catheters and medical instruments including catheters. In one embodiment, there is provided a medical instrument comprising a base with a port for connection to an infusion set, a catheter extending from the base, and comprises a main body of tubular configuration, a plurality of lateral conduits distributed along the length of the catheter, an elastomeric cover over each lateral conduit, said elastomeric cover opens with a fluid flow inside the catheter and closes when the fluid flow stops. Said lateral conduits have an angle in the range of 15° to 60° to the longitudinal axis of the catheter.

In one embodiment, wherein the lateral conduits have an opposite direction to the fluid flow inside the catheter.

In one embodiment successive lateral conduits in the longitudinal direction of the catheter are at different longitudinal lines and/or at different circumferential positions of the catheter.

In one embodiment the lateral conduits have different angles to the longitudinal axis of the catheter.

In one embodiment least one lateral conduit extends in a direction having a larger proximal component than at least one lateral conduit which is located more distally on the catheter.

In one embodiment, the catheter comprises a proximal portion without lateral conduits.

In one embodiment, the catheter comprises a tip terminating in a distal tip opening and a trocar extends through the tip opening, and the tip opening is configured to collapse closed upon withdrawal of the trocar.

In another embodiment, there is provided A catheter comprising a main body of tubular configuration, a plurality of lateral conduits distributed along the length of the catheter, an elastomeric cover over each lateral conduit, said elastomeric cover opens with a fluid flow inside the catheter and closes when the fluid flow stops. Said lateral conduits have an angle in the range of 15° to 60° to the longitudinal axis of the catheter, and directed in opposite direction of the fluid flow inside the catheter.

### BRIEF DESCRIPTION OF DRAWINGS

Described herein is a catheter and a medical instrument including a catheter. The description and accompanying figures, which describe and show certain embodiments, are made to demonstrate, in a nonlimiting manner, several possible configurations of medical instrument, catheters etc. of using them according to various aspects and features of the present disclosure. Accordingly, the disclosure is not limited to the specific embodiments described. The invention will be better understood, and objects other than those set forth above will become apparent when consideration is given to the following detailed description thereof. Such description makes reference to the drawings wherein:

FIG. 1 is a view of the present invention of the vascular catheter inserted in a vessel;

FIG. 2 is a perspective view of a medical instrument of the invention;

FIG. 3 is a cut-away perspective view along part of the length of the catheter;

FIG. 4 is a longitudinal sectional view of the vascular catheter;

FIG. 5 is a pair of longitudinal sectional views showing the withdrawal of the trocar at a distal end of the catheter and automatic closing of the tip opening;

FIG. 6 is a longitudinal sectional diagram illustrating the flow of infusion liquid in the catheter,

FIG. 7 is a diagram showing infusion flows from the catheter in the context of the vessel.

FIG. 8 shows enlarged, encircled sectional views of the elastomeric cover in the open (2b) and closed (3b) position of the present invention above the display of the vascular catheter inserted in a vessel.

FIG. 9: Flow rates through different openings of the 15-degree catheter.

FIG. 10 Flow rates through different openings of the 30-degree catheter.

FIG. 11: Flow rates through different openings of the 45-degree catheter.

FIG. 12: Flow rates through different openings of the 60-degree catheter.

FIG. 13: Magnified saline flow for traditional, and ASRF catheter at 15° 511 (b), 30° (c), 45° (d), and 60° (e). Note the reduced area of stagnation at the angle of entry 512 (circled) in ASRF catheter compared to the traditional catheter.

### DETAILED DESCRIPTION

The new design of the anti-thrombotic vascular retrograde flow catheter is made to be used in a reverse mode of operation than the previous designs for vascular catheter prior art, making the design useful and practical and can include an elastomeric cover over the lateral conduit opens with the fluid flow and closes when the fluid flow stops. The elastomeric cover over the lateral conduit opens with the fluid flow and closes when the fluid flow stops. The aim of the elastomeric cover (valve like) is to ensure an anti-stasis retrograde flow of fluids and to prevent blood from stasis (and subsequently clotting) inside the catheter when the catheter not in use i.e. no flow through the lateral conduits.

According to some embodiments, the flow-diverting catheter is adapted to create an initial anti-stasis retrograde flow and minimize the risk of peri-catheter stasis, and catheter-related thrombosis (CRT). An anti-thrombotic vascular retrograde flow catheter has a proximal infusion inlet with a number of infusion fluid outflow lateral conduits in the catheter wall extending in a direction with a proximal component. The backward direction of each lateral conduit is in the range of 10° to 90° to the proximal longitudinal. Hence, instead of the infusion fluid flowing distally from the tip, it flows proximally against the antegrade blood flow and in doing so helps to prevent stasis-induced thrombosis from forming around the catheter. The vascular catheter can be made from polyvinyl chloride, Teflon, hydrogel material, silicone, rubber or the like. The design of the catheter will significantly reduce catheter-related thrombosis which is associated with many sequalae and complications as catheter failure, thrombophlebitis, pulmonary embolism, longer hospital stay, increased patients' morbidity and mortality and increased health care cost.

According to the invention, there is provided a vascular catheter device comprising a catheter with a proximal infusion inlet and an outlet distally of the inlet in a longitudinal direction, wherein the outlet includes at least one infusion fluid outflow lateral conduit extending in a direction with a proximal component. An object of the invention is to have an effective method of preventing fibrin sheath resulting in bacterial formation, infection etc. and reduce burden for health care workers and help establish a better recovery period for patients being treated for various illnesses. The retrograde flow will help to prevent peri-catheter stasis and catheter-related thrombosis (CRT) in patients.

A preferred embodiment of the present invention will include an elastomeric cover version and have a collapsible tip made of an elastomeric material (a polymer with viscoelasticity (having both viscosity and elasticity) and very weak inter-molecular forces, generally having low Young's modulus and high failure strain compared with other materials). The tip is preferably characterized by: (a) naturally collapsing after removal of the trocar from within the catheter; (b) can be reopened by re-introducing the trocar or by a very high pressure of fluid within the catheter's lumen. In alternative embodiments, the present invention vascular catheter device will not have an elastomeric cover. An alternative embodiment over the lateral conduit opens with the fluid flow and closes when the fluid flow stops. There may be lateral conduits in a pattern around the circumference, with at least some lateral conduits longitudinally offset. A vascular catheter device wherein the lateral conduit extends in a direction in the range of 10° to 90° to a proximal longitudinal direction. The direction of the lateral conduits 10° is approximately 45° to longitudinal.

In other embodiments, the angle may be in the range of 10° to 90°, and preferably 15° to 60°, and more preferably 15° to 45°, as set out in more detail below. Some of the lateral conduits may have different axial directions. For example, more distal conduits preferably have directions which have larger angles to longitudinal, i.e. have a smaller proximal component than more proximal conduits. This is because there is a greater need for proximal flow closer to the point of entry of the catheter into the blood vessel, in order to prevent stasis-induced thrombosis. Also, more distal conduits preferably are smaller than more proximal conduits. This is for the same reason of achieving more proximal flow closer to the point of entry into the vessel. Because the lateral conduits introduce a flow restriction as compared to the conventional distal tip opening, the extent of the lateral conduit direction proximal component should only be that required to minimize the risk of thrombosis and not more. The lateral conduits are configured to be present in the catheter in a way that does not compromise the other mechanical properties of the catheter, such as kink resistance. They are sized and distributed to avoid undesirable loss of strength according to overall configuration. By way of background, the typical length of a peripheral vascular catheter ranges from 19 mm (pediatric) to 45 mm (adult). The typical length of a central vascular catheter (that can be inserted in the internal jugular vein, subclavian vein or femoral vein) ranges from 50 mm to 150 mm for pediatric patients and ranges from 130 mm to 700 mm for adult patients. The thickness of the wall of the peripheral vascular catheter ranges from 0.3 mm to 1 mm, with increasing the length of the catheter the manufacturer increases the thickness of the wall of the catheter to decrease the risk of kinking. The lateral conduits preferably have an angle in the range of 15° to 60° to the longitudinal axis of the catheter on the proximal side. At the lower end of the range the infusion flow has a larger directional component along the catheter wall, and towards the upper end of the range the more the infusion flow will be towards the vascular wall. It is envisaged that there may be, in the one catheter, a pattern of lateral conduits with different angles at different longitudinal and/or peripheral positions to achieve a desired effect of impingement on the vessel wall and having a desired proximal component in direction. For example, there may be alternate groups of one or more lateral conduit at 15° and 60° with respect to longitudinal in the same catheter. The alternating pattern may be circumferential or longitudinal. The length of each lateral conduit is set primarily by (a) the thickness of the wall of the catheter, which in turn depends on the length of the catheter and the degree of kink resistance required, and (b) the angle of the lateral conduit, in which the lesser the angle, the longer will be the lateral conduit. The number of the lateral conduits is according to the desired flow rate from the catheter, i.e. increasing the number of openings will increase the fluid flow rate through catheter. The distribution of the lateral conduit will depend on their number, among other factors. The distribution pattern may be such that successive openings in the longitudinal direction are not along a same longitudinal line. For example, each alternate lateral conduit may be circumferentially offset by 90° from the previous opening. This may provide one or more spiral patterns along the length of the catheter. It may provide a zigzag pattern in side view, with each second opening being on a different one of two longitudinal lines at 90° or other angle separation. Irrespective of the pattern, it is preferred that, to minimize the risk of kinking no single longitudinal position has multiple lateral conduits, and most preferably diametrically opposed openings are avoided. It is also preferred that lateral conduits at different circumferential positions be provided to achieve the desired proximal flow effects of reduced thrombosis all around the catheter. The angle of the lumen direction of the lateral conduits and holes can be chosen to vary (different angle degrees) in order to make the retrograde flow adjacent to or away from the outer surface in different grades towards the inner surface of the vessel catheterized. The same catheter can have their lateral conduits with either the same angles of direction of their lumens or with different angles of direction of their lumens. The self-closing tip opening can be modified to be either (a) absent as in case of catheter insertion through a sheath or (b) can be present without self-closing property but with a narrower diameter helping enforcing the fluid flow through the side holes or openings with a retrograde direction of their lumens and subsequently flow.

In overview, the side holes or openings in the catheter are preferably characterized by the following, in various embodiments: The lateral conduits are preferably higher in number than the openings in prior art conventional catheters in order to allow for both (a) better flow rate by increasing the total surface area available for infused fluid to flow through, and (b) to provide a flow with a proximal component along the base and the shaft of the catheter to prevent blood stasis along the whole length of the catheter. The lateral conduits may be distributed along the length of the catheter and at different levels around the circumference of the catheter in order to provide the proximal flow around the catheter at all levels and in all directions. The number and the distribution of the lateral conduits (longitudinal and circumferential distributions) can be chosen according to the required rate of the fluid infusion. This may be a directly proportional relationship. The internal diameter of each lateral conduit along its length within the wall of the catheter is preferably uniform in order to achieve adequate fluid flow through it and decrease resistance to fluid flow. The tip opening of the catheter is characterized by the following, in various embodiments: In case of vascular catheters that are inserted by the help of a metal trocar inside the vascular catheter (as are most peripheral vascular catheters) the tip opening is configured to collapse and close after removal of the trocar from inside of the catheter in order to help directing the fluid flowing through the catheter to flow through the lateral conduits in order to produce the targeted retrograde flow. The same principle will be applied to catheters inserted by the Seldinger technique (as in most central vascular catheters) in which the tip opening will collapse and close after removal of the guide wire from inside the catheter to enable directing the flow through the lateral conduits. In case of vascular catheters that are inserted through a guide sheath, the tip opening may be absent (blinded-tip catheter) allowing the infused fluid to flow only in a retrograde direction through the backward directed lateral conduit. A conventional catheter for sheath insertion without a trocar typically has a distal opening. However, in the invention, there is no need for such an opening because there is no need for a trocar.

The features of the above-described embodiments, but without a distal opening or trocar, apply to this embodiment. The device may be used in any of the conventional manners by the medical personnel, in the antegrade direction of blood flow. Any of these methods are tailored to provide the desired flow rate of infusion fluid through the catheter, and it is envisaged that an allowance will be required to accommodate the fact that there might be more flow resistance, depending on the number and size of the lateral conduits. These methods include: Gravity feed to enforce fluid to flow from fluid bag through an infusion set or connection then through a cannula to the bloodstream in the vessel, by making the fluid bag situated at a higher level than the level of the body, the higher the level of the bag the higher the fluid flow rate through the cannula. A much less common method depends on applying a pressure bag with a manometer, the pressure bag surrounding and compressing the fluid bag to force fluid to flow at a maximum possible rate. This method is used mainly for rapid resuscitation of patients with massive bleeding and in need for rapid and large fluid replacement. A method using an infusion pump or infusion syringe that is electrically operated and contains a small computer processor to control the flow of fluid by infusing a certain volume over a certain time. This type is used mainly in intensive care units for accurate calculation of volume infused over time. In summary, the above methods will provide a force (gravity or hydrostatic pressure, extrinsic pressure of pressure bag, or pressure created by electric pump or syringe) and that force will push the fluid through the vascular catheter and out through the side openings or conduits. Any of these forces will push the fluid through the side openings or conduits and as the long axis of the conduits is directed backwards the fluid will flow initially in a retrograde direction preventing stasis and thrombosis before losing force and flowing in the antegrade flow towards the heart. The invention can be applied to both peripheral and central vascular catheters. The catheter provides a retrograde flow of the infused fluid with maintaining the antegrade technique of the catheter insertion that is often used by medical personnel. The retrograde flow of fluids infused will decrease blood stasis in the area between the catheter and the vessel and subsequently will decrease catheter-related thrombosis and its sequences as superficial or deep thrombosis, possible pulmonary embolism, thrombophlebitis and catheter obstruction or failure. Thus, the invention can allow a longer lifespan of the vascular catheters with lower complications related to catheter-associated thrombosis, and this will preserve vessels of patients, decrease the discomfort of patients, decrease the load on medical staff, and will benefit patients who have poor vascular access or a lengthy hospital stay. The invention may be applied to any of a range of vascular access treatments for the administration of medications, blood, or blood products. The catheter may be inserted into different types of vessels, both peripheral and central, including internal jugular veins, subclavian veins, or femoral veins. It helps to prevent stasis of the blood at the angle between the vascular wall and the antegrade catheter (i.e. catheters inserted with the direction of the blood flow), thereby preventing precipitation of thrombosis and thrombus formation initially at the angle and then around the catheter. It avoids a situation in which thrombosis can extend around the catheter reaching its tip of extending beyond the catheter's tip. This thrombosis can be the initiative of venous thrombosis with subsequent complications as superficial and deep venous thrombosis and possible pulmonary embolism. The present invention can be applied to either intravenous or intra-arterial vascular catheters, taking into consideration the differences in the direction of blood flow and the possible sites of stasis and subsequently stasis that can mandate changes in the number, site, size, level, direction and position of the lateral conduits or holes in order to decrease blood stasis and thrombosis and subsequently to increase durability (lifespan) of the catheter and decrease complications related to stagnation and thrombosis. The invention is not limited to the embodiments described but may be varied in construction and detail. It is envisaged that one or more lateral conduit may protrude from the catheter main body wall for closer impingement towards a vessel wall and/or for more proximal flow direction.

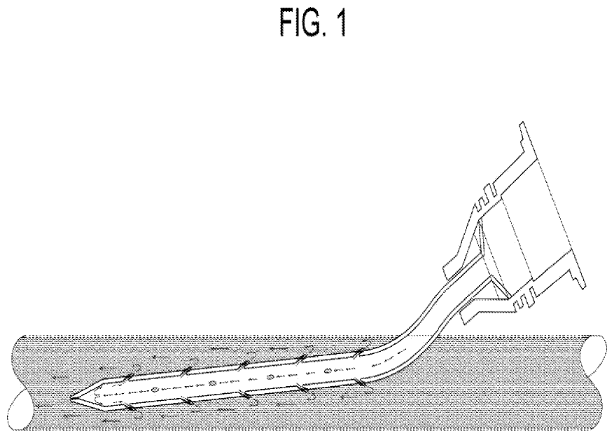

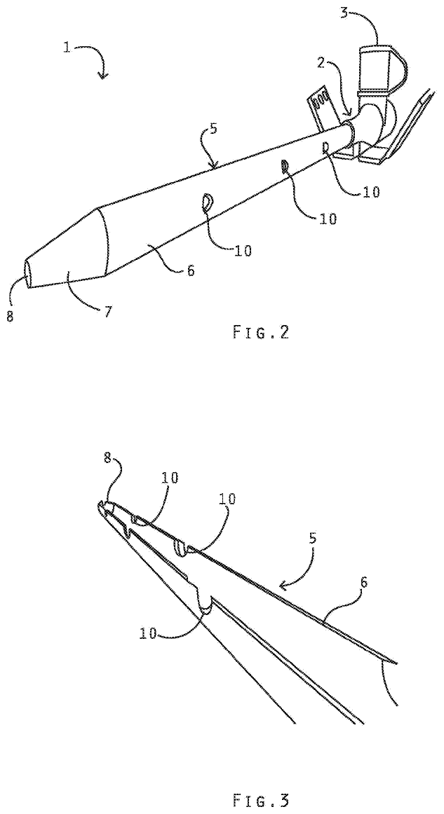

Referring to FIG. 1 displays how the present invention can be inserted into a blood vessel. FIGS. 2 to 4 a medical instrument comprising a vascular or venous catheter 1, a base 2 with a port 3 for connection to an infusion set. In this specification, the base is defined as being proximal, and the distal direction is along the longitudinal axis of the catheter from the base towards the tip. A catheter 5 extends from the base 2, and comprises a main body 6 of tubular configuration and a tip 7 terminating in a distal tip opening 8. A trocar 15 extends through the catheter 5 and has a tip extending distally of the catheter tip opening 8. The catheter 5 further comprises six lateral conduits 10, three on each diagonally opposed side. However, there is only one conduit at each longitudinal position, as otherwise there might be a risk of kinking by an excessive weakening of the catheter main body 6 at a particular location. Each lateral conduit 10 extends radially and proximally for outflow of infusion liquid in a direction having a proximal component. Each lateral conduit 10 is a small conduit formed through the wall of the catheter main body 6, the conduit having an axial direction with a proximal component set by the angle of the conduit with respect to the longitudinal axis of the catheter. In this embodiment, the direction of the lateral conduits 10 is approximately 45° to longitudinal.

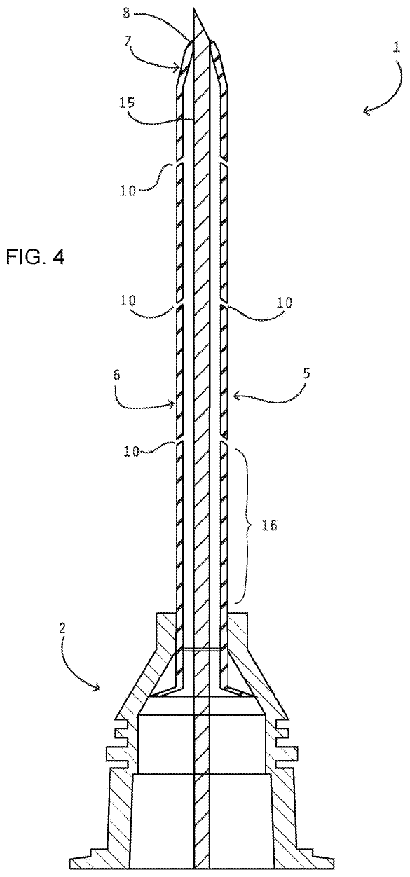

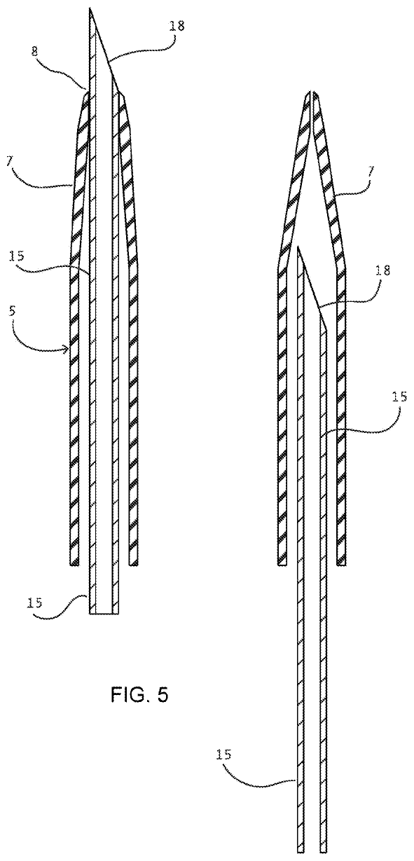

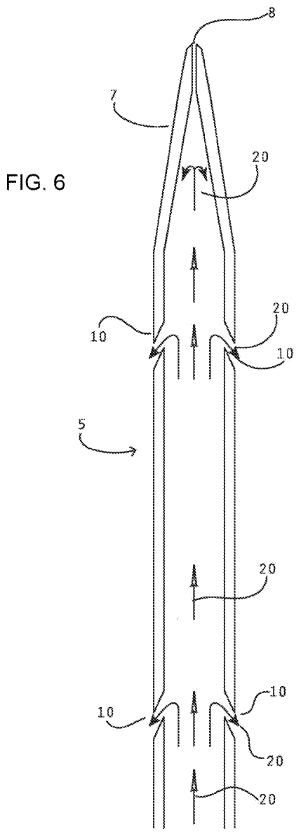

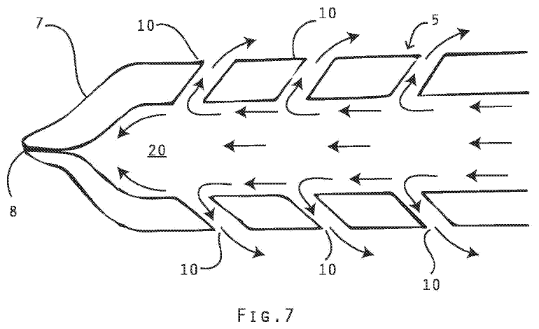

As shown in FIG. 4 the catheter main body 6 has a proximal portion 16 without lateral conduits, for passing through the skin, tissue, and the vessel wall. This section preferably has a minimum length of: 3 mm in peripheral vascular catheters for pediatrics (total catheter length is typically 19 mm in smallest catheters), 6 mm in peripheral vascular catheters for adult patients (total catheter length is typically 45 mm in largest), 3 cm in central vascular catheters for pediatrics (total catheter length is typically 8 cm in smallest catheters), or 6 cm in central vascular catheters for adults (total catheter length typically is 13 cm in the smallest catheter). In use, as shown in FIG. 5 after the catheter insertion (FIG. 5(a)) the trocar 15 is withdrawn. The tip 7 material is configured to close over (FIG. 5(b)) due to natural resilience when the trocar 15 is withdrawn. The collapsible tip 7 prevents fluid from flowing through the opening 8 and directs the infusion fluid to flow through the lateral conduits 10 to provide retrograde flow around the catheter (for antegrade insertion). Upon connection of the infusion set, infusion liquid flows as shown by the arrows 20 in FIG. 6. This flow is not out through the distal end of the catheter as would be conventional, rather only out through the lateral conduits 10. Due to the directions of these openings, which have the form of short conduits, the infusion liquid exits the catheter in a direction both radially and proximally. This is very different from the prior art conventional flow as shown in FIG. 1, solely distal flow from the tip.

Referring to FIG. 7 the beneficial effect of this flow is illustrated. The region 25 downstream (in terms of blood flow) of the catheter entry receives a flow of infusion liquid in the retrograde direction, meeting the (antegrade) blood flow B. Hence, there is less risk of thrombus developing due to stasis. Blood stasis and thrombosis around the catheter and between the catheter and the vessel is a serious problem, however, the backward directed lateral conduits in the catheter can provide a flow of fluids infused in order to decrease blood stasis and thrombosis in the region between the catheter and the vessel thus decreasing catheter associated thrombosis and subsequent complications. This avoids the need for the clinician to insert the catheter in the retrograde (against blood flow) direction to minimize the risk of thrombus at the axilla. It is in general preferable that insertion is in the antegrade direction, and clinicians are much more familiar with the method.

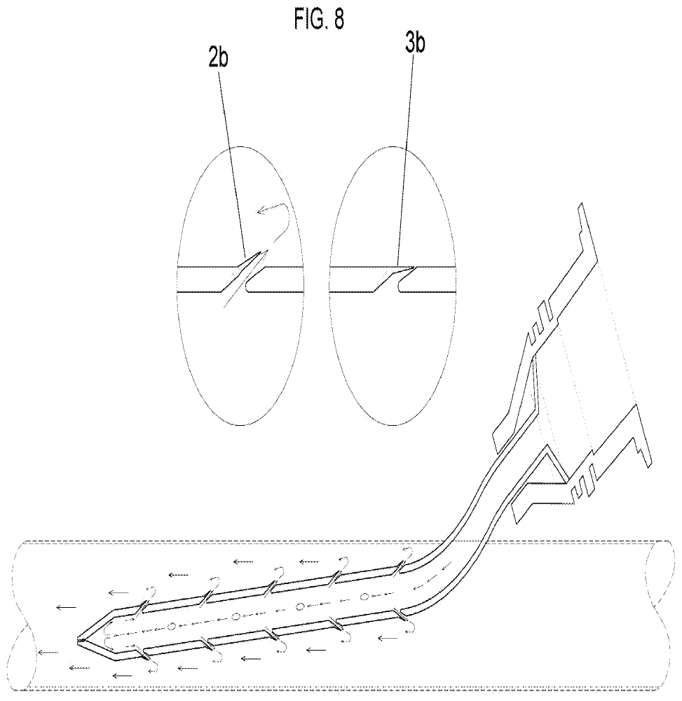

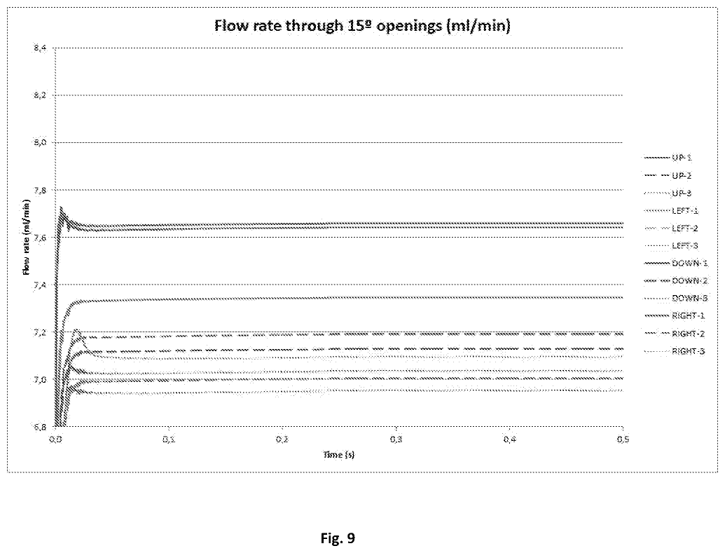

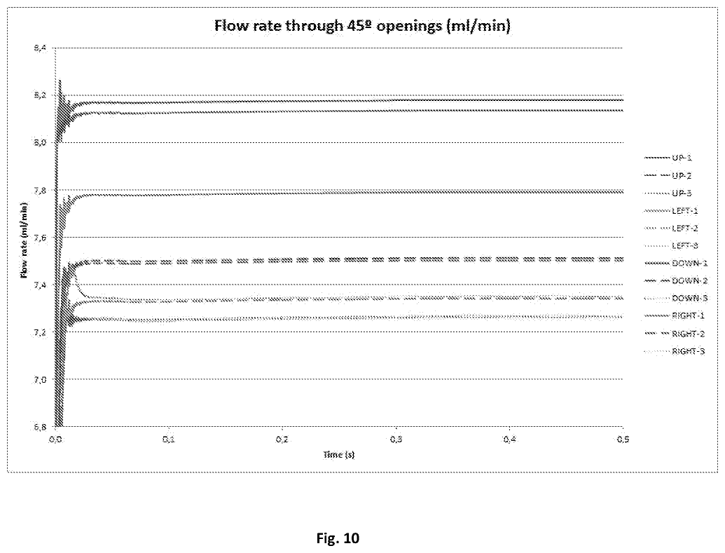

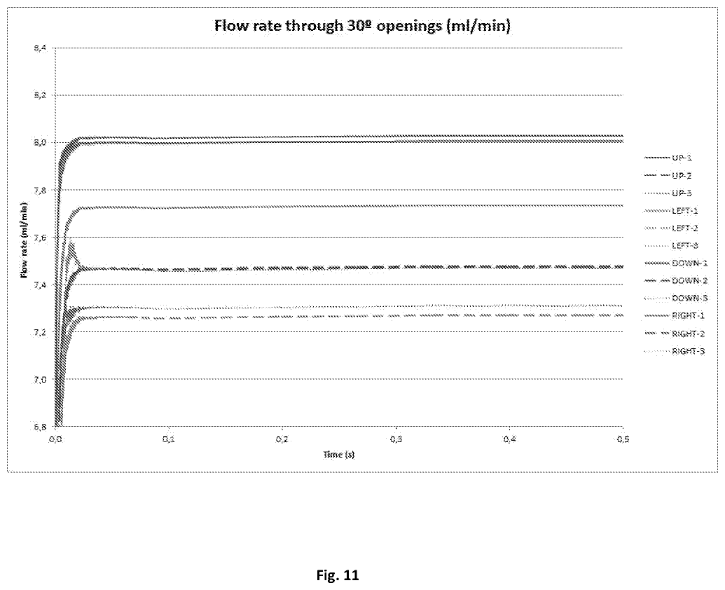

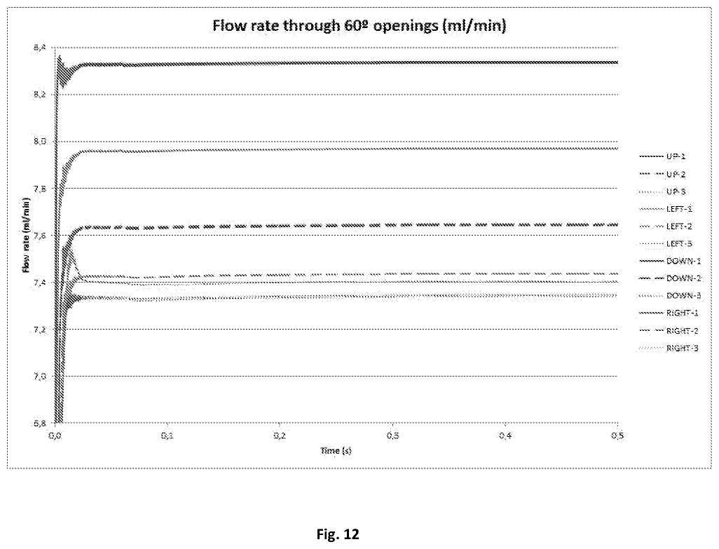

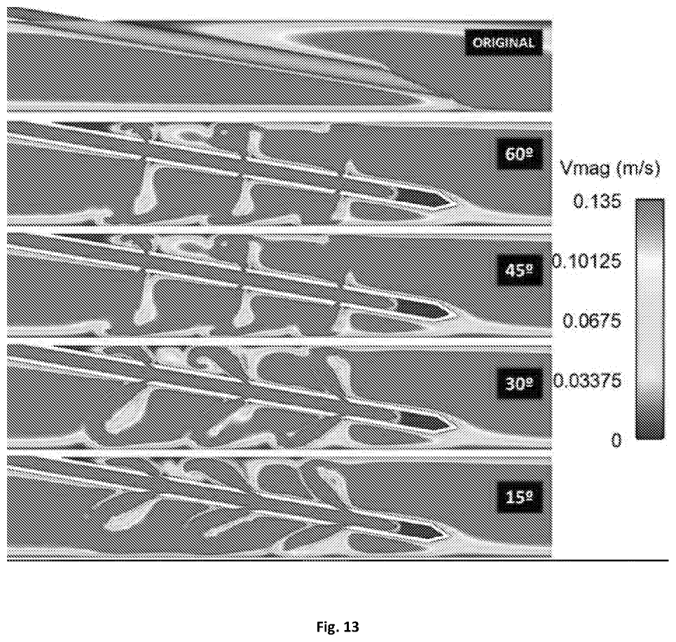

FIG. 8 shows what the cover's function does. It displays enlarged, encircled sectional views of the elastomeric cover in the open (2b) and closed (3b) position of the present invention above the image of the vascular catheter being inserted in a vessel.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. An antithrombotic medical instrument comprising:
a base (2) with a port (3) for connection to an infusion set;
a vascular catheter (5) extending from the base (2), and comprises a main body (6) of tubular configuration and a collapsible tip made of an elastomeric material, wherein there is a distal opening in said tip configured to be opened by a trocar that extends in use through the tip opening and to collapse closed upon withdrawal of the trocar;
a plurality of lateral conduits distributed along the length of the catheter;
an elastomeric cover over each lateral conduit, said elastomeric cover opens with a fluid flow inside the catheter and closes when the fluid flow stops;
said antithrombotic medical instrument characterised in that the lateral conduits are distributed longitudinally and circumferentially around the catheter, have an angle in the range of 15° to 60° to the longitudinal axis of the catheter, and directed in opposite direction of a fluid flow inside the catheter to provide a retrograde fluid flow around the catheter.

2. (canceled)

3. A medical instrument according to claim 1 wherein successive lateral conduits in the longitudinal direction of the catheter are at different longitudinal lines.

4. A medical instrument according to claim 1 wherein lateral conduits are at different circumferential positions of the catheter.

5. A medical instrument according to claim 4 wherein the lateral conduits have different angles to the longitudinal axis of the catheter.

6. (canceled)

7. A medical instrument as claimed in claim 1, wherein the catheter comprises a proximal portion without lateral conduits.

8. A medical instrument according to claim 7, wherein said proximal catheter portion has a minimum length of 3 mm.

9. (canceled)

10. (canceled)

11. (canceled)

12. (canceled)
